(19) United States
(12) Patent Application Publication
(10) Pub. No.: US 2020/0146582 A1
(43) Pub. Date: May 14, 2020

(54) ELECTROCORTICOGRAPHIC ELECTRODE, BRAIN ACTIVITY ACQUISITION AND CONTROL SYSTEM, AND BRAIN ACTIVITY ACQUISITION AND CONTROL METHOD

(71) Applicant: RIKEN, WAKO-SHI (JP)

(72) Inventor: Misako KOMATSU, (US)

(73) Assignee: RIKEN, Wako-shi (JP)

(21) Appl. No.: 16/681,409

(22) Filed: Nov. 12, 2019

(30) Foreign Application Priority Data
Nov. 9, 2018 (JP) 2018-210975

(51) Int. Cl. A61B 5/0484; G06F 3/01; A61B 5/00; A61B 5/0478

(52) U.S. Cl. CPC A61B 5/0484; A61B 5/0478; A61B 5/0006; G06F 3/015

(57) ABSTRACT

The electrocorticographic electrode includes a first connector unit including a connector, the connector having a plurality of connection terminals electrically connectable to the plurality of wirings; one or more electrode units extending in a first direction and having a base connected to the first connector unit; an electrode unit for inferior division of temporal lobe extending in a second direction intersecting the first direction via a wiring unit, the wiring unit extending in the first direction and having a base connected to the first connector unit; a ground electrode unit connectable to ground potential and having a base connected to the first connector unit; and a reference electrode unit detectable a reference signal and having a base connected to the first connector unit, wherein an electrode unit for superior division of temporal lobe is provided on at least one end of the one or more electrode units, the electrode unit for superior division of temporal lobe extending in the second direction and including an electrode that can be placed in proximity to an electrode arranged in the electrode unit for inferior division of temporal lobe.

### CROSS-REFERENCE TO RELATED APPLICATION

This application is based upon and claims the benefit of priority from Japanese Patent Application No. 2018-210975, filed Nov. 9, 2018; the entire contents of which are incorporated herein by reference.

### FIELD

The disclose relates to an electrocorticographic electrode, a brain activity acquisition and control system, and a brain activity acquisition and control method.

### BACKGROUND

In recent years, research on BMI (Brain Machine Interface) technology has been actively conducted all over the world. In the BMI technology, information on brain activity is read out from a brain by connecting the brain of a living body and the external device. By using the read-out information on brain activity, an intention of the living body can be predicted (also called “decoding”), or a device can be controlled according to its prediction result. Thereby, for example, the living body can control a device substituting for a certain site by remote control only by imaging the action of the site with the brain. Such BMI technology is raised expectation for practical use as a technology capable of reconstructing motor functions lost due to accidents or illnesses, cognitive sensory functions, communication functions, or the like, for example.

Regarding BMI technology, methods for acquiring information on brain activity at a plurality of positions on the cerebral cortex by placing non-inserting type electrocorticographic (hereinafter referred to as ECoG) electrodes on the surface of the cerebral cortex or methods for applying photostimulation to the cerebral cortex by placing non-inserting type ECoG electrodes and a LED array on the surface of the cerebral cortex are known. These methods are disclosed in Japanese Unexamined Patent Application Publication No. 2014-1233329, “Facilitative effect of repetitive presentation of one stimulus on cortical responses to other stimuli in macaque monkeys—a possible neural mechanism for mismatch negativity” (Takakura, K. and Fujii, N., European Journal of Neuroscience, Nov. 27, 2015, Vol. 43, pp. 516-528), “Mismatch negativity in common marmosets: Whole-cortical recordings with multi-channel electrocorticograms” (Komatsu, M., Takakura, K. and Fujii, N., SCIENTIFIC REPORTS, Oct. 12, 2015, 5:15006, doi: 10.1038/srep15006, and “An electrocorticographic electrode array for simultaneous recording from medial, lateral, and intrasulcal surface of the cortex in macaque monkeys” (Fukushima, M., Saunders, R., C, Mullarkey, M., Doyle, A., M., Mishkin, M., Fujii, N., Journal of Neuroscience Methods, Aug. 15, 2014, 233:155-165).

### SUMMARY

The first aspect of some embodiments is an electrocorticographic electrode in which a plurality of electrodes that can be placed at a plurality of positions of a cerebral cortex and a plurality of wirings electrically connected to each of the plurality of electrodes are arranged on a deformable substrate. The electrocorticographic electrode includes a first connector unit including a connector, the connector having a plurality of connection terminals electrically connectable to the plurality of wirings; one or more electrode units extending in a first direction and having a base connected to the first connector unit; an electrode unit for inferior division of temporal lobe extending in a second direction intersecting the first direction via a wiring unit, the wiring unit extending in the first direction and having a base connected to the first connector unit; a ground electrode unit connectable to ground potential and having a base connected to the first connector unit; and a reference electrode unit detectable a reference signal and having a base connected to the first connector unit, wherein an electrode unit for superior division of temporal lobe is provided on at least one end of the one or more electrode units, the electrode unit for superior division of temporal lobe extending in the second direction and including an electrode that can be placed in proximity to an electrode arranged in the electrode unit for inferior division of temporal lobe.

The second aspect of some embodiments is an electrocorticographic electrode in which a plurality of electrodes that can be placed at a plurality of positions of a cerebral cortex and a plurality of wirings electrically connected to each of the plurality of electrodes are arranged on a deformable substrate. The electrocorticographic electrode includes a second connector unit including a connector, the connector having a plurality of connection terminals electrically connectable to the plurality of wirings, an electrode unit for visual cortex having a base connected to the second connector unit; an electrode unit for visual dorsal stream extending in a third direction and having a base connected to the second connector unit; an electrode unit for occipital pole extending in the third direction and having a base connected to the second connector unit, an electrode unit for visual ventral stream extending in a fourth direction intersecting the third direction and having a base connected to an end of the electrode unit for visual dorsal stream; a ground electrode unit connectable to ground potential and having a base connected to the second connector unit; and a reference electrode unit detectable a reference signal and having a base connected to the second connector unit.

The third aspect of some embodiments is an electrocorticographic electrode including; a photostimulation electrode including a plurality of light sources and a connector unit, the plurality of light sources being capable of irradiating a plurality of positions in an anterior part of a cerebral cortex with light, the connector unit including a connector having a plurality of terminals for controlling the light sources electrically connected to the light sources; an electrocorticographic electrode for anterior part including the electrocorticographic electrode of the first aspect; an electrocorticographic electrode for posterior part including the electrocorticographic electrode of the second aspect; a case member configured to accommodate the first connector unit, the second connector unit, and the connector unit of the photostimulation electrode so as to be capable of being held, the first connector unit, the second connector unit, and the connector unit of the photostimulation electrode being stacked so as not to overlap each other, and in which an opening is formed so that at least the plurality of connection terminals of the first connector unit, the plurality of connection terminals of the second connector unit, and the plurality of terminals for controlling the plurality of light sources are exposed; and a cover member configured to cover the opening formed in the case member.

The fourth aspect of some embodiments is a brain activity acquisition and control system including: any one of the electrocorticographic electrode of described above; a light emission controller configured to control the plurality of light sources; and a recording controller configured to record electrocorticographic signals detected through the plurality of electrodes in a storage.

The fifth aspect of some embodiments is a brain activity acquisition and control system including: any one of the electrocorticographic electrode described above; a light emission controller configured to control the plurality of light sources based on electrocorticographic signals detected through the plurality of electrodes.

The sixth aspect of some embodiments is a brain activity acquisition and control method including: a light emission control step of controlling the plurality of light sources; and a recoding control step of recording electrocorticographic signals detected through the plurality of electrodes of any one of the electrocorticographic electrode described above in a storage.

The seventh aspect of some embodiments is a brain activity acquisition and control method including: a detection step of detecting electrocorticographic signals through the plurality of electrodes of any one of the electrocorticographic electrode described above; and a light emission control step of controlling the plurality of light sources based on the electrocorticographic signals detected in the detection step.

### BRIEF DESCRIPTION OF THE DRAWINGS

FIG. 1 is a schematic diagram illustrating an example of the configuration of an ECoG electrode according to embodiments.

FIG. 2 is a schematic diagram illustrating an example of the configuration of an ECoG electrode according to the embodiments.

FIG. 3 is a schematic diagram illustrating an example of the configuration of an ECoG electrode according to the embodiments.

FIG. 4 is a schematic diagram illustrating an example of the configuration of an ECoG electrode according to the embodiments.

FIG. 5 is a schematic diagram illustrating an example of the configuration of an ECoG electrode according to the embodiments.

FIG. 6 is a schematic diagram illustrating an example of the configuration of an ECoG electrode according to the embodiments.

FIG. 7 is a schematic diagram illustrating an example of the configuration of an ECoG electrode according to the embodiments.

FIG. 8 is a schematic diagram illustrating an example of the configuration of an ECoG electrode according to the embodiments.

FIG. 9 is a schematic diagram illustrating an example of the configuration of an ECoG electrode according to the embodiments.

FIG. 10 is a schematic diagram illustrating an example of the configuration of an ECoG electrode according to the embodiments.

FIG. 11 is a schematic diagram illustrating an example of the configuration of an ECoG electrode according to the embodiments.

FIG. 12 is a schematic diagram illustrating an example of the configuration of an ECoG electrode according to the embodiments.

FIG. 13 is a schematic diagram illustrating an example of the configuration of an ECoG electrode according to the embodiments.

FIG. 14 is a schematic diagram illustrating an example of the configuration of an ECoG electrode according to the embodiments.

FIG. 15 is a schematic diagram illustrating an example of a method of forming the ECoG electrode according to the embodiments.

FIG. 16 is a schematic diagram illustrating an example of the state of mounting the ECoG electrode according to the embodiments.

FIG. 17 is a schematic diagram illustrating an example of the state of mounting the ECoG electrode according to the embodiments.

FIG. 18 is a schematic diagram illustrating an example of the state of mounting the ECoG electrode according to the embodiments.

FIG. 19 is a schematic diagram illustrating an example of the configuration of a brain activity acquisition and control system according to the embodiments.

FIG. 20 is a schematic diagram illustrating an example of the operation of the brain activity acquisition and control system according to the embodiments.

FIG. 21 is a schematic diagram illustrating an example of the operation of the brain activity acquisition and control system according to the embodiments.

### DETAILED DESCRIPTION

In the ECoG electrodes known to the inventors, it is difficult to arrange electrodes at desired positions according to the size and shape of the cerebrum of a living body. Therefore, there were various problems such as large noise superimposed on the neural signals due to the movement of the living body.

According to the following embodiments, a new technique for acquiring information on brain activity of a cerebrum cortex of a living body or performing photostimulation on the cerebrum cortex can be provided.

Exemplary embodiments of an electrocorticographic (ECoG) electrode (ECoG electrodes), a brain activity acquisition and control system (brain activity processing system), and a brain activity acquisition and control method (brain activity processing method) according to the present invention will be described in detail with reference to the drawings. Any of the contents of the documents cited in the present specification and arbitrary known techniques may be applied to the embodiments below.

An ECoG electrode according to embodiments includes a deformable flexible substrate. One or more electrodes (thin film electrodes) that can be placed at one or more positions on the cerebral cortex and one or more wirings electrically connected to the one or more electrodes on (in) the flexible substrate. The flexible substrate is provided with a connector unit on which a connector for electrically connecting to an external device is mounted. The connector includes a plurality of connection terminals and is electrically connected to the one or more electrodes via the one or more wires described above. The ECoG electrode according to the embodiments includes a case member and a cover member. The case member is configured to accommodate the connector unit so as to be capable of being held. In the case member, an opening is formed so that at least the connection terminals are exposed. The cover member is configured to cover (occlude) the opening formed in the case member. The case member is arranged on the skull of a living body while accommodating the connector unit so as to be capable of being held.

The ECoG electrode according to the embodiments includes a photostimulation electrode. The photostimulation electrode also includes a flexible substrate. One or more light sources (for example, LED (Light Emitting Diode) light sources) that can be placed at one or more positions on the cerebral cortex and one or more wirings electrically connected to the one or more electrodes on (in) the flexible substrate. The flexible substrate is provided with a connector unit on which a connector for electrically connecting to an external device is mounted. The connector includes a plurality of terminals for controlling the light sources and is electrically connected to the one or more light sources describe above via the one or more wires described above. By using the photostimulation electrode, ECoG signals (neural signals, information on brain activity) at a plurality of positions can be simultaneously measured while applying a photostimulation to the plurality of positions of the cerebral cortex.

The ECoG electrode according to the embodiments can simultaneously measure ECoG signals at a plurality of positions throughout the cerebral cortex of the hemisphere (left hemisphere and right hemisphere) of the cerebrum of the living body.

The ECoG electrode according to the embodiments includes an ECoG electrode for left hemisphere and an ECoG electrode for right hemisphere, and can simultaneously measure ECoG signals at a plurality of positions throughout the cerebral cortex of the left hemisphere or the right hemisphere using either ECoG electrode. The entire cerebral cortex of the entire hemisphere (left hemisphere and right hemisphere) of the cerebrum of the living body is covered by combining the ECoG electrode for left hemisphere and the ECoG electrode for right hemisphere. Thereby, ECoG signals at a plurality of positions throughout the entire cerebral cortex of the entire hemisphere can be simultaneously measured.

The ECoG electrode for left hemisphere according to the embodiments includes an ECoG electrode for anterior part of the cerebral cortex and an ECoG electrode for posterior part of the cerebral cortex, and can simultaneously measure ECoG signals at a plurality of positions on the anterior part or the posterior part using either ECoG electrode. The entire cerebral cortex of the left hemisphere of the cerebrum of the living body is covered by combining the ECoG electrode for anterior part of the left hemisphere and the ECoG electrode for posterior part of the left hemisphere. Thereby, ECoG signals at a plurality of positions throughout the entire cerebral cortex of the left hemisphere can be simultaneously measured.

In the same way, the ECoG electrode for right hemisphere according to the embodiments includes an ECoG electrode for anterior part of the cerebral cortex and an ECoG electrode for posterior part of the cerebral cortex, and can simultaneously measure ECoG signals at a plurality of positions on the anterior part or the posterior part using either ECoG electrode. The entire cerebral cortex of the right hemisphere of the cerebrum of the living body is covered by combining the ECoG electrode for anterior part of the right hemisphere and the ECoG electrode for posterior part of the right hemisphere. Thereby, ECoG signals at a plurality of positions throughout the entire cerebral cortex of the right hemisphere can be simultaneously measured.

Hereinafter, the anterior part of the cerebral cortex according to the embodiments will be described as a region including the prefrontal lobe, the temporal lobe, and a part of the parietal lobe. And the posterior part of the cerebral cortex according to the embodiments will be described as a region including the occipital lobe and the region including the remaining part of the parietal lobe. However, the anterior part and the posterior part of the cerebral cortex according to the embodiments are not limited to these. A part of the posterior part may be included in the anterior part, or a part of the anterior part may be included in the posterior part.

Hereinafter, an ECoG electrode applicable to a cerebrum of a small living body, a brain activity acquisition and control system using this ECoG electrode, and the like will be described. Examples of the small living body include marmosets belonging to the small primates, and the like. The cerebrum of the marmoset has higher cerebral functions similar to those of humans and has few cerebral sulci. Thereby, the cerebrum of the marmoset is suitable for measuring information on brain activity in the cerebral cortex.

### [ECog Electrode]

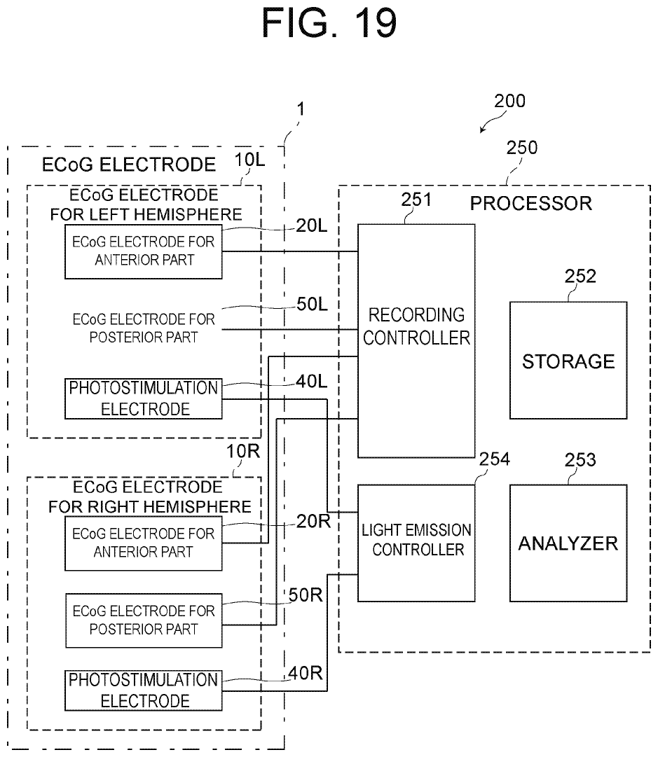

An ECoG electrode 1 (for example, see FIG. 19) according to the embodiments includes an ECoG electrode 10L for left hemisphere and an ECoG electrode 10R for right hemisphere. The ECoG electrode 10L includes a plurality of electrodes and a plurality of wirings, as described above. The configuration of the ECoG electrode 10R is the same as the configuration of the ECoG electrode 10L. A plurality of electrodes and a plurality of wirings in the ECoG electrode 10R are mirror-arranged with respect to a plurality of electrodes and a plurality of wirings in the ECoG electrode 10L.

Hereinafter, the configuration of the ECoG electrode 10L for left hemisphere will be mainly described. For the configuration of the ECoG electrode 10R for right hemisphere, “L” at the end of the explanation of the ECoG electrode 10L may be read as “R”, for example.

### <For Left Hemisphere>

The ECoG electrode 10L for left hemisphere includes an ECoG electrode 20L for anterior part, a photostimulation electrode 40L, and an ECoG electrode 50L for posterior part.

### (ECoG Electrode 20L for Anterior Part)

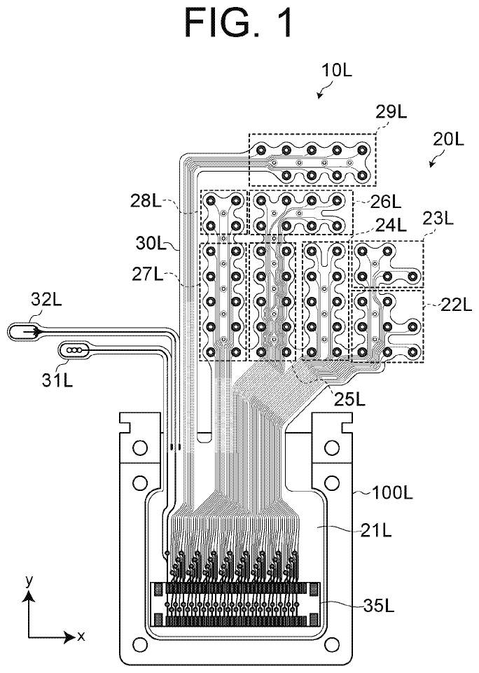

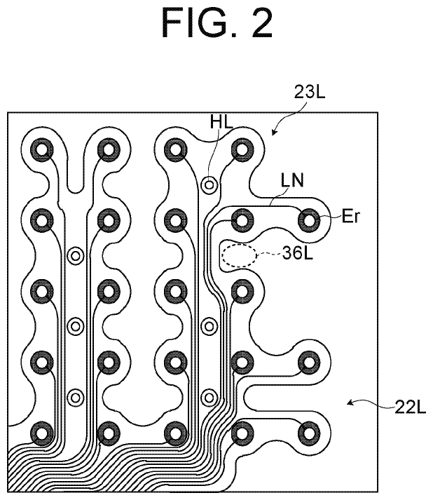

FIG. 1 and FIG. 2 illustrate examples of the configuration of the ECoG electrode 20L for anterior part according to the embodiments. FIG. 1 represents a top view of the ECoG electrode 20L for anterior part. FIG. 2 schematically represents an enlarged view of FIG. 1. In FIG. 2, like reference numerals designate like parts as in FIG. 1. The same description may not be repeated.

The ECoG electrode 20L for anterior part includes a first connector unit 21L, a plurality of electrode units, a ground electrode unit 31L, and a reference electrode unit 32L. Each of the electrode units is provided with one or more electrodes (thin film electrodes) (for example, an electrode Er in FIG. 2) provided on a part of the outer edge part and one or more conductive wirings (for example, a wiring LN in FIG. 2) electrically connected to each of the one or more electrodes.

A connector 35L is mounted on the first connector unit 21L. The connector 35L has a plurality of connection terminals configured to electrically connect to a plurality of wirings provided in each of the plurality of electrode units, the ground electrode unit 31L, and the reference electrode unit 32L. The first connector unit 21L is fixed to a case member 100L as described later. In some embodiments, the first connector unit 21L is provided on a rigid substrate.

Each of the plurality of electrode units includes a plurality of electrodes and a plurality of wirings. The plurality of electrodes and the plurality of wirings are used for detecting ECoG signals at a plurality of measurement sites on each cerebral lobe of the anterior part of the left hemisphere of the cerebral cortex. Each of the plurality of electrodes units has a base connected to the first connector unit 21L and is formed so as to extend substantially in a y direction (first direction). As described later, in order to increase the range that can be irradiated the cerebral cortex with light from above the electrode without being blocked by the ECoG electrode 20L for anterior part, the outer edge part of each electrode unit is formed along the shape of the electrode. Further, one or more transmissive parts (for example, transmissive holes) (for example, hole HL in FIG. 2) are formed in the each electrode unit so that light from the one or more LED light sources provided on the photostimulation electrode 40L is transmitted. The photostimulation electrode 40L is described later and is stacked on the ECoG electrode 20L for anterior part.

The plurality of electrode units include an electrode unit 22L for prefrontal cortex, an electrode unit 23L for orbitorfrontal cortex, a first electrode unit 24L for prefrontal lobe, a second electrode unit 25L for prefrontal lobe, electrode units 26L and 28L for superior division of temporal lobe, and an electrode unit 27L for parietal lobe.

The electrode unit 22L for prefrontal cortex has a base connected to the first connector unit 21L and includes one or more wirings extending substantially in they direction and one or more electrodes electrically connected to the one or more wirings. The one or more electrodes can be placed at the prefrontal cortex. At least one of the one or more electrodes can be placed at the frontal pole.

The electrode unit 23L for orbitorfrontal cortex has a base connected to the electrode unit 22L for prefrontal cortex and includes one or more wirings and one or more electrodes electrically connected to the one or more wirings. The one or more electrodes can be placed at the orbitorfrontal cortex.

As shown in FIG. 2, a constricted portion (in the broad sense, a cutout portion) 36L is formed at an outer edge part between the electrode unit 22L for prefrontal cortex and the electrode unit 23L for orbitorfrontal cortex. In this manner, the degree of freedom in bending the substrate can be improved by forming the constricted portion 36L. Thereby, the one or more electrodes can be placed with high accuracy without causing the substrate to deflect or the like in a portion where the shape changes in a curved shape in the range from the prefrontal cortex to the orbitorfrontal cortex. In particular, the one or more electrodes can be placed at a desired position on the orbitorfrontal cortex by folding the substrate in the orbitorfrontal cortex located in the inferior region of the cerebral cortex.

The first electrode unit 24L for prefrontal lobe has a base connected to the first connector unit 21L and includes one or more wirings extending substantially in they direction and one or more electrodes electrically connected to the one or more wirings. The one or more electrodes can be placed at the prefrontal lobe.

The second electrode unit 25L for prefrontal lobe has a base connected to the first connector unit 21L and includes one or more wirings extending substantially in they direction and one or more electrodes electrically connected to the one or more wirings. The one or more electrodes can be placed at the prefrontal lobe. The electrode unit 26L for superior division of temporal lobe is connected to an end of the second electrode unit 25L for prefrontal lobe. The electrode unit 26L for superior division of temporal lobe includes one or more wirings extending substantially in a x direction (second direction) and one or more electrodes electrically connected to the one or more wirings. The one or more electrodes can be placed at the superior division of temporal lobe.

One end of the electrode unit 27L for parietal lobe is connected to the first connector unit 21L. The electrode unit 27L for parietal lobe includes one or more wirings extending substantially in the y direction and one or more electrodes electrically connected to the one ore more wirings. The one or more electrodes can be placed at the parietal lobe. In some embodiments, the electrode unit 28L for superior division of temporal lobe is connected to the other end of the electrode unit 27L for parietal lobe. The electrode unit 28L for superior division of temporal lobe includes one or more wirings and one or more electrodes electrically connected to the one or more wirings. The one or more electrodes can be placed at the superior division of temporal lobe.

Further, the ECoG electrode 20L for anterior part includes an electrode unit 29L for inferior division of temporal lobe. The electrode unit 29L for inferior division of temporal lobe extends substantially in the x direction via a wiring unit 30L. The wiring unit 30L has a base connected to the first connector unit 21L and extends substantially in the y direction. The electrode unit 29L for inferior division of temporal lobe includes one or more wirings and one or more electrodes electrically connected to the one or more wirings. The one or more electrodes can be placed at the inferior division of temporal lobe. That is, in order to detect ECoG signals of temporal lobe, the electrode unit 29L for inferior division of temporal lobe is provided through the wiring unit 30L separately from the electrode unit 26L (28L) for superior division of temporal lobe. And the electrodes of the electrode unit 26L (28L) for superior division of temporal lobe are placed in proximity to the electrodes arranged on the electrode unit 29L for inferior division of temporal lobe. Thereby, the electrode unit 26L (28L) for superior division of temporal lobe can be placed from upward of the temporal lobe (superior division of the temporal lobe). And the electrode unit 29L for inferior division of temporal lobe can be placed from downward of the temporal lobe (inferior division of the temporal lobe). Therefore, the brain activity in the temporal lobe of the small cerebral cortex can be measured with high density.

The ground electrode unit 31L has a base connected to the first connector unit 21L and includes an electrode for connecting a ground potential. The electrode of the ground electrode unit 31L can be installed, for example, outside the skull of the living body, and is electrically connected to a predetermined position outside the skull. The ground potential is a reference potential for the ECoG signals detected using each of the electrodes described above and a reference signal described later.

The reference electrode unit 32L has a base connected to the first connector unit 21L and includes an electrode for detecting the reference signal. The electrode of the reference electrode unit 32L can be installed, for example, inside the skull of the living body, and is electrically connected to a predetermined position inside the skull. The reference signal is a signal used as a reference for the ECoG signals detected using each of the electrodes described above. On both the reference signal and the ECoG signals, substantially similar noise is superimposed. Thereby, the true signal component(s) can be extracted by subtracting the reference signal from the ECoG signals.

Each of the plurality of electrodes provided on the ECoG electrode 20L for anterior part is electrically connected to a plurality of connection terminals provided on the connector 35L through a corresponding wiring.

The case member 100L is configured to accommodate the first connector unit 21L so as to be capable of being held. The upper part of the connector 35L mounted on the first connector unit 21L can be covered with a cover member (not shown). Thereby, when measuring the brain activity of the living body, the cover member can be removed, and an adapter of a cable connecting the external device can be connected to the connector 35L, and the external device can be electrically connected using the cable. Thus, the connector 35L fixed by the case member 100L is arranged on the skull of the living body, and the ECoG electrode 20L for anterior part and the external device (not shown) are connected using the cable via the connector 35L during measurement. Therefore, the breakage of the cable due to the movement of the living body can be prevented. And the information on brain activity can be measured for a long time from the awakening living body.

### (Photostimulation Electrode 40L)

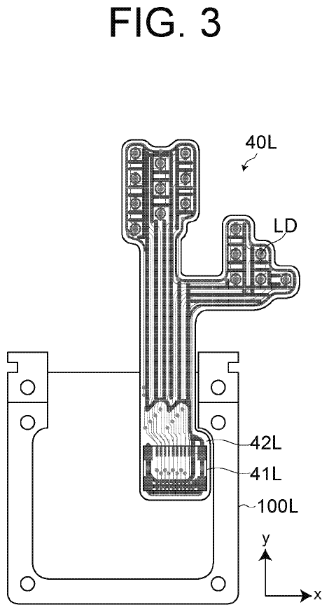

FIG. 3 shows an example of the configuration of the photostimulation electrode 40L according to the embodiments. FIG. 3 represents a top view of the photostimulation electrode 40L. The photostimulation electrode 40L is held by the case member 100L in FIG. 1. In FIG. 3, like reference numerals designate like parts as in FIG. 1, and the redundant explanation may be omitted as appropriate.

The photostimulation electrode 40L includes a plurality LED light sources (LED light source shown in FIG. 3) and a connector unit 42L. The plurality of LED light sources is capable of irradiating a plurality of positions in at least an anterior part of the cerebral cortex with light. The connector unit 42L is provided with a connector 41L. The connector 41L includes a plurality of terminals for controlling the light sources electrically connected to the plurality of LED light sources.

Each of the plurality of LED light sources provided on the photostimulation electrode 40L is electrically connected the plurality of terminals for controlling light sources provided on the connector 41L through a corresponding wiring. A desired LED light source can be turned on by applying a current or voltage to the terminal for controlling light source from the external device (not shown) via the connector 41L.

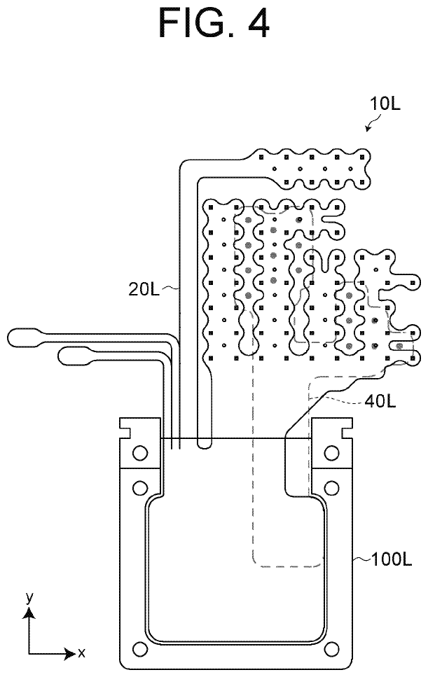

FIG. 4 shows an example of the configuration in the case of combining the ECoG electrode 20L for anterior part and the photostimulation electrode 40L. In FIG. 4, like reference numerals designate like parts as in FIG. 1 and FIG. 3. The same description may not be repeated.

The ECoG electrode 20L for anterior part and the photostimulation electrode 40L are stacked in the main surface direction so that the mounting areas of the connectors do not overlap. In the case member 100L, the information on brain activity can be measured while applying a photostimulation to the anterior part of the left hemisphere of the cerebral cortex by stacking the ECoG electrode 20L for anterior part shown in FIG. 1 and the photostimulation electrode 40L shown in FIG. 3. The photostimulation electrode 40L is stacked above the ECoG electrode 20L for anterior part so that the ECoG electrode 20L for anterior part is arranged between the photostimulation electrode 40L and the cerebral cortex. Thereby, the ECoG signals of the anterior part of the cerebral cortex can be detected at high densities. As shown in FIG. 1 and FIG. 2, the transmissive holes (hole HL in FIG. 2) are formed at the positions corresponding to the plurality of LED light sources provided on the photostimulation electrode 40L in the ECoG electrode 20L for anterior part. For example, the transmissive holes are formed between the wirings. In some embodiments, a transmissive member is provided at a position where the transmissive hole is formed.

### (ECoG Electrode 50L for Posterior Part)

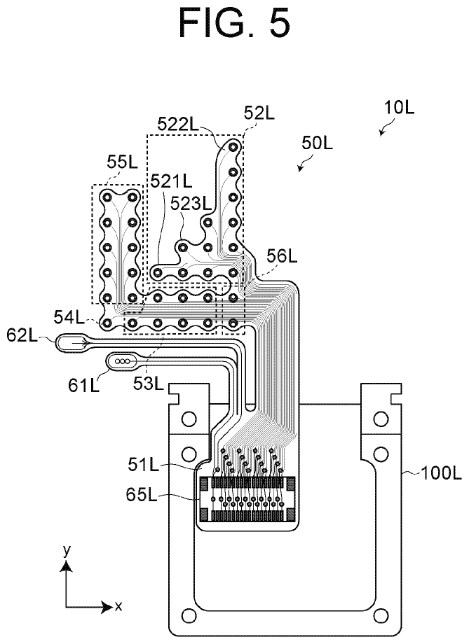

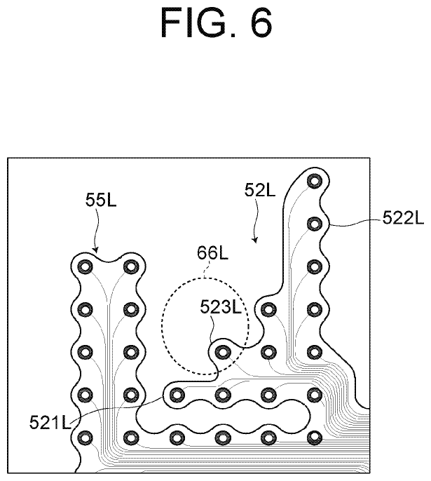

FIG. 5 and FIG. 6 illustrate examples of the configuration of the ECoG electrode SOL for posterior part according to the embodiments. FIG. 5 represents a top view of the ECoG electrode SOL for posterior part. FIG. 6 schematically represents an enlarged view of FIG. 5. In FIG. 6, like reference numerals designate like parts as in FIG. 5. The same description may not be repeated.

The ECoG electrode SOL for posterior part includes a second connector unit 51L, a plurality of electrode units, a ground electrode unit 61L, and a reference electrode unit 62L. Similar to the ECoG electrode 20L for anterior part, each of the electrode units is provided with one or more electrodes (thin film electrodes) provided on a part of the outer edge part and one or more conductive wirings electrically connected to each of the one or more electrodes.

A connector 65L is mounted on the second connector unit 51L. The connector 65L has a plurality of electrodes and a plurality of connection terminals configured to electrically connect to a plurality of wirings provided in each of the plurality of electrode units, the ground electrode unit 61L, and the reference electrode unit 62L. The second connector unit 51L is fixed to the case member 100L as described later. In some embodiments, the second connector unit 51L is provided on a rigid substrate.

Each of the plurality of electrode units includes a plurality of electrodes and a plurality of wirings. The plurality of electrodes and the plurality of wirings are used for detecting ECoG signals at a plurality of measurement sites on each cerebral lobe of the posterior part of the left hemisphere of the cerebral cortex. Each of the plurality of electrodes has a base connected to the second connector unit 51L. In some embodiments, similar to the ECoG electrode 20L for anterior part, the outer edge part of each electrode unit is formed along the shape of the electrode. In some embodiments, one or more transmissive parts (for example, transmissive holes) are formed in the each electrode unit so that light from the one or more LED light sources provided on the photostimulation electrode 40L is transmitted. The photostimulation electrode 40L is described later and is stacked on the ECoG electrode 50L for posterior part.

The plurality of electrode units includes an electrode unit 52L for visual cortex, an electrode unit 53L for visual dorsal stream, an electrode unit 54L for occipital pole, an electrode unit 55L for visual ventral stream, and an electrode unit 56L for parietal lobe.

The electrode unit 52L for visual cortex has a base connected to the second connector unit 51L and includes one or more wirings extending substantially in the y direction (fourth direction) and one or more electrodes electrically connected to the one or more wirings. The one or more electrodes can be placed at the visual cortex.

The electrode unit 53L for visual dorsal stream has a base connected to the second connector unit 51L and includes one or more wirings extending substantially in the x direction (third direction) and one or more electrodes electrically connected to the one or more wirings. The one or more electrodes can be placed at the visual dorsal stream.

The electrode unit 54L for occipital pole has a base connected to the second connector unit 51L and includes one or more wirings extending substantially in the x direction and one or more electrodes electrically connected to the one or more wirings. The one or more electrodes can be placed at the occipital pole. In some embodiments, the electrode unit 54L for occipital pole is connected to an end of the electrode unit 53L for visual dorsal stream.

The electrode unit 55L for visual ventral stream has a base connected to the end of the electrode unit 53L for visual dorsal stream and includes one or more wirings extending substantially in the y direction and one or more electrodes electrically connected to the one or more wirings. The one or more electrodes can be placed at the visual ventral stream.

One end of the electrode unit 56L for parietal lobe is connected to the second connector unit 51L. The electrode unit 56L for parietal lobe includes one or more wirings extending substantially in the x direction and one or more electrodes electrically connected to the one ore more wirings. The one or more electrodes can be placed at the parietal lobe.

The electrode unit 52L for visual cortex includes a first electrode unit 521L for visual cortex, a second electrode unit 522L for visual cortex, and a third electrode unit 523L for visual cortex. The first electrode unit 521L for visual cortex includes one or more wirings extending substantially in the x direction and one or more electrodes electrically connected to the one or more wirings. The second electrode unit 522L for visual cortex includes one or more wirings extending substantially in the y direction and one or more electrodes electrically connected to the one or more wirings. The third electrode unit 523L for visual cortex includes one or more electrodes formed in a region (gap portion 66L shown in FIG. 6) partially surrounded by the first electrode unit 521L for visual cortex and the second electrode unit 522L for visual cortex. As shown in FIG. 6, the gap portion 66L is formed between the electrode unit 52L for visual cortex and the electrode unit 55L for visual ventral stream by forming the electrode unit 52L for visual cortex in this manner. By forming the gap portion 66L, the degree of freedom in bending the substrate can be improved. Thereby, the one or more electrodes can be placed with high accuracy without causing the substrate to deflect or the like in a portion where the shape changes in a curved shape in the range from the visual cortex to the visual ventral stream.

The ground electrode unit 61L has a base connected to the second connector unit 51L and includes an electrode for connecting the ground potential. Similar to the ground electrode unit 31L, the electrode of the ground electrode unit 61L can be installed, for example, outside the skull of the living body, and is electrically connected to a predetermined position outside the skull.

The reference electrode unit 62L has a base connected to the second connector unit 51L and includes an electrode for detecting the reference signal. Similar to the reference electrode unit 32L, the electrode of the reference electrode unit 62L can be installed, for example, inside the skull of the living body, and is electrically connected to a predetermined position inside the skull.

Each of the plurality of electrodes provided on the ECoG electrode SOL for posterior part is electrically connected to a plurality of connection terminals provided on the connector 65L through a corresponding wiring.

The ECoG electrode 20L for anterior part, the photostimulation electrode 40L, and the ECoG electrode SOL for posterior part are stacked so that the mounting areas of the connectors do not overlap. At this time, the second connector unit 51L is accommodated by the case member 100L so as to be capable of being held. The upper part of the connector 65L mounted on the second connector unit 51L can be covered with a cover member (not shown). Thereby, when measuring the brain activity of the living body, the cover member can be removed, and an adapter of a cable connecting the external device can be connected to the connector 65L, and the external device can be electrically connected using the cable. Thus, the connector 65L fixed by the case member 100L is arranged on the skull of the living body, and the ECoG electrode 20L for anterior part and the external device (not shown) are connected using the cable via the connector 65L during measurement. Therefore, the breakage of the cable due to the movement of the living body can be prevented. And the information on brain activity can be measured for a long time from the awakening living body.

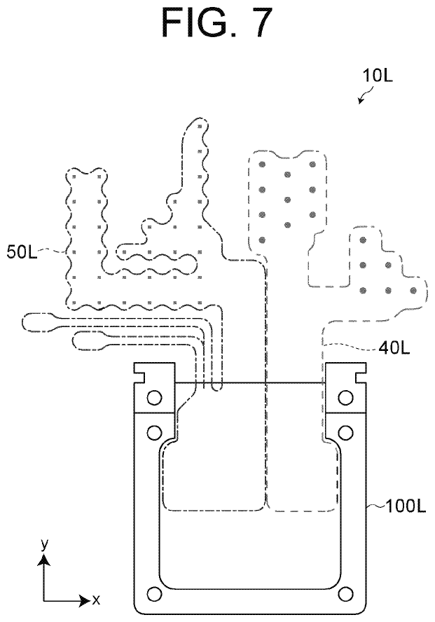

FIG. 7 shows an example of the configuration in the case of combining the ECoG electrode SOL for posterior part and the photostimulation electrode 40L. In FIG. 7, like reference numerals designate like parts as in FIG. 3 and FIG. 5. The same description may not be repeated.

In the case member 100L, the information on brain activity can be measured while applying a photostimulation to the cerebral cortex by stacking the ECoG electrode SOL for posterior part shown in FIG. 5 and the photostimulation electrode 40L shown in FIG. 3. The photostimulation electrode 40L is stacked above the ECoG electrode SOL for posterior part so that the ECoG electrode SOL for posterior part is arranged between the photostimulation electrode 40L and the cerebral cortex. In some embodiments, the transmissive holes are formed in the ECoG electrode SOL for posterior part at the positions corresponding to the plurality of LED light sources provided on the photostimulation electrode 40L.

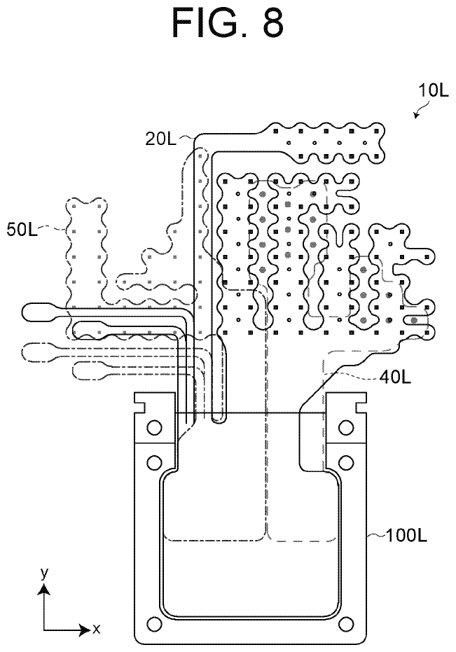

FIG. 8 shows an example of the configuration in the case of combining the ECoG electrode 20L for anterior part, the ECoG electrode SOL for posterior part and the photostimulation electrode 40L. In FIG. 8, like reference numerals designate like parts as in FIG. 1, FIG. 3, and FIG. 5. The same description may not be repeated.

In the case member 100L, the information on brain activity of the anterior part and the posterior part of the left hemisphere can be measured while applying a photostimulation to the cerebral cortex by stacking the ECoG electrode 20L for anterior part shown in FIG. 1, the ECoG electrode 50L for posterior part shown in FIG. 5, and the photostimulation electrode 40L shown in FIG. 3. The photostimulation electrode 40L is stacked above the ECoG electrode 20L for anterior part and the ECoG electrode 50L for posterior part so that the ECoG electrode 20L for anterior part and the ECoG electrode 50L for posterior part are arranged between the photostimulation electrode 40L and the cerebral cortex.

That is, the case member 100L accommodates the ECoG electrode 20L for anterior part, the ECoG electrode 50L for posterior part, and the photostimulation electrode 40L by holding the first connector unit 21L and the second connector unit 51L, and the connector unit 42L of the photostimulation electrode 40L. The ECoG electrode 20L for anterior part, the ECoG electrode 50L for posterior part, and the photostimulation electrode 40L are stacked so as not to overlap each other. An opening is formed in the case member 100L so that at least the plurality of connection terminals of the first connector unit 21L, the plurality of connection terminals of the second connector unit 51L, and the plurality of terminals for controlling the plurality of light sources are exposed. The opening can be covered using a cover member (not shown).

### <For Right Hemisphere>

Similar to the ECoG electrode 10L for left hemisphere, the ECoG electrode 1 OR for right hemisphere includes an ECoG electrode 20R for anterior part, a photostimulation electrode 40R, and an ECoG electrode 50R for posterior part.

### (ECoG Electrode 20R for Anterior Part)

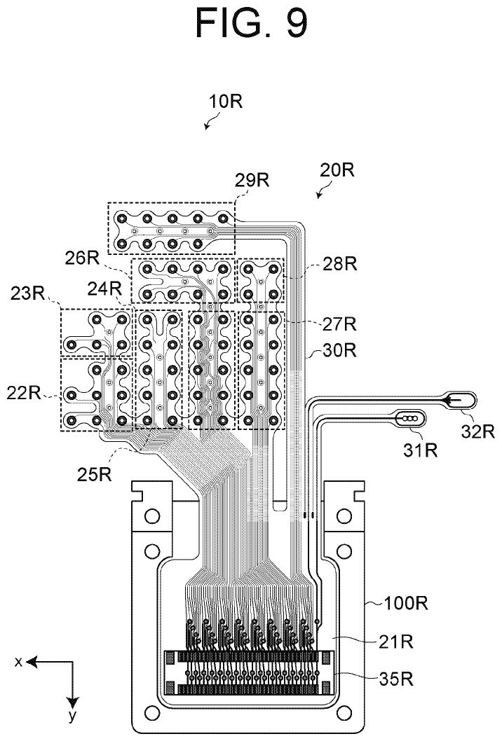

FIG. 9 illustrate an example of the configuration of the ECoG electrode 20R for anterior part according to the embodiments. The configuration of the ECoG electrode 20R for anterior part is the same as the configuration of the ECoG electrode 20L for anterior part except that the electrodes and the wirings of the ECoG electrode 20R for anterior part are mirror-arranged with respect to the electrodes and the wirings of the ECoG electrode 20L for anterior part shown in FIGS. 1 and 2. Therefore, the description is omitted.

### (Photostimulation Electrode 40R)

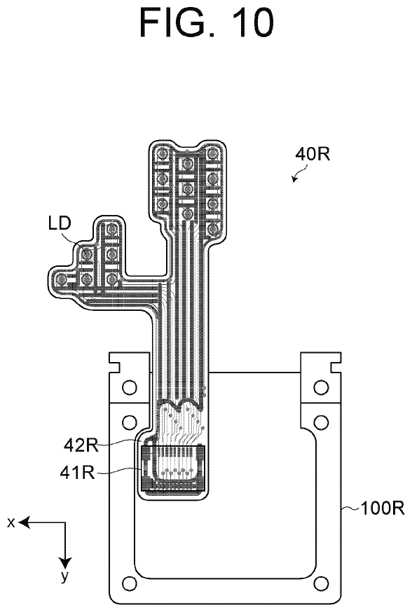

FIG. 10 shows an example of the configuration of the photostimulation electrode 40R according to the embodiments. The configuration of the photostimulation electrode 40R is the same as the configuration of the photostimulation electrode 40L except that the electrodes and the wirings of the photostimulation electrode 40R are mirror-arranged with respect to the electrodes and the wirings of the photostimulation electrode 40L shown in FIG. 3. Therefore, the description is omitted.

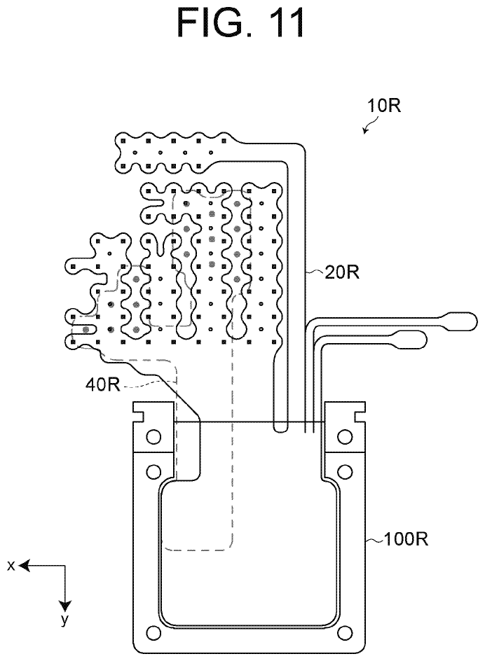

FIG. 11 shows an example of the configuration in the case of combining the ECoG electrode 20R for anterior part and the photostimulation electrode 40R. In FIG. 11, like reference numerals designate like parts as in FIG. 9 and FIG. 10. The same description may not be repeated. In FIG. 11, the configuration shown in FIG. 11 is the same as the configuration shown in FIG. 4 except that the electrodes and the wirings shown in FIG. 11 are mirror-arranged with respect to the electrodes and the wirings shown in FIG. 4. Therefore, the description is omitted.

### (ECoG Electrode 50R for Posterior Part)

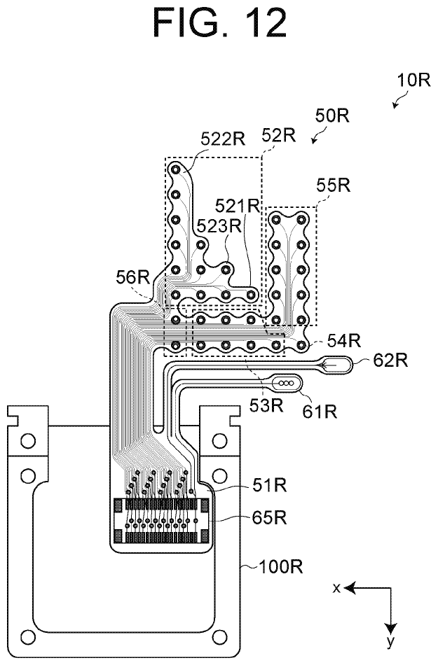

FIG. 12 illustrates an example of the configuration of the ECoG electrode 50R for posterior part according to the embodiments. The configuration of the ECoG electrode 50R for posterior part is the same as the configuration of the ECoG electrode 50L for posterior part except that the electrodes and the wirings of the ECoG electrode 50R for posterior part are mirror-arranged with respect to the electrodes and the wirings of the ECoG electrode 50L for posterior part shown in FIGS. 5 and 6. Therefore, the description is omitted.

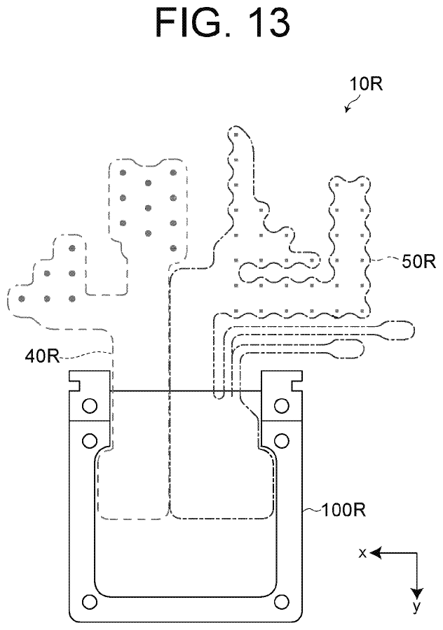

FIG. 13 shows an example of the configuration in the case of combining the ECoG electrode 50R for posterior part and the photostimulation electrode 40R. In FIG. 13, like reference numerals designate like parts as in FIGS. 10 and 12. The same description may not be repeated. In FIG. 13, the configuration shown in FIG. 13 is the same as the configuration shown in FIG. 7 except that the electrodes and the wirings shown in FIG. 13 are mirror-arranged with respect to the electrodes and the wirings shown in FIG. 7. Therefore, the description is omitted.

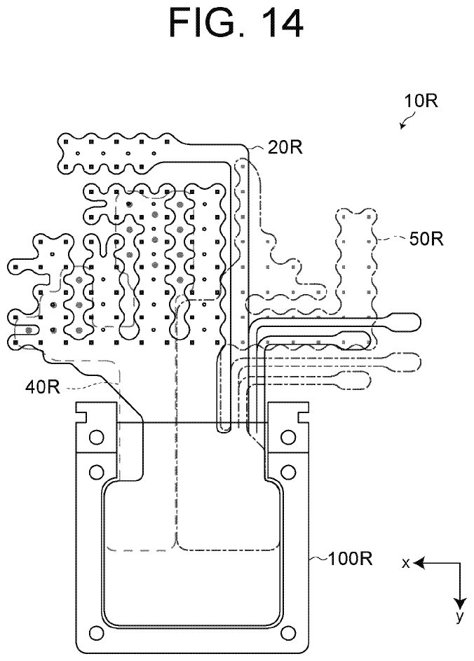

FIG. 14 shows an example of the configuration in the case of combining the ECoG electrode 20R for anterior part, the ECoG electrode 50R for posterior part and the photostimulation electrode 40R. In FIG. 14, like reference numerals designate like parts as in FIGS. 9, 10, and 12. The same description may not be repeated. In FIG. 14, the configuration shown in FIG. 14 is the same as the configuration shown in FIG. 8 except that the electrodes and the wirings shown in FIG. 14 are mirror-arranged with respect to the electrodes and the wirings shown in FIG. 8. Therefore, the description is omitted.

### <Method of Forming ECoG Electrode>

The ECoG electrode according to the embodiments can be formed as follows.

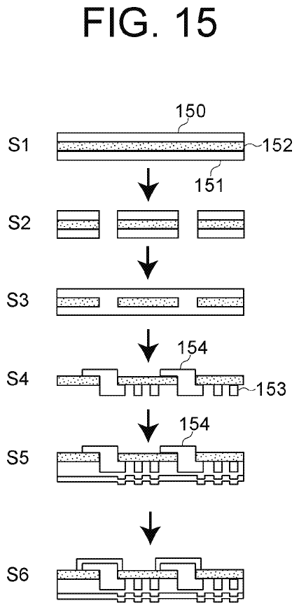

FIG. 15 shows an explanatory diagram of an example of method of forming the ECoG electrode according to the embodiments.

First, a flexible substrate 152 is prepared (S1). The flexible substrate 152 is made of polyimide resin or the like having copper layers 150 and 151 as conductive layers formed on the front surface and the back surface. The copper layers 150 and 151 are examples of conductive materials formed on the flexible substrate 152. The polyimide resin is an example of a non-conductive material.

Next, hole parts are formed by laser drilling at the positions where the electrodes are placed on the flexible substrate 152 (S2).

Subsequently, the front surface and the back surface are conducted through the hole parts formed in step S2 by performing copper plating on the front surface and the back surface (S3).

The electrodes 154 and the wirings 153 are formed performing an etching process on the copper layers 150 and 151 formed on the front surface and the back surface (S4).

Next, insulating processing is performed on the wirings 153 by pasting a polyimide film in the layer on which the wirings 153 are formed using an adhesive or the like (S5).

Finally, gold plating is performed on the electrodes 154 (S6).

### <Method of Placing on Living Body>

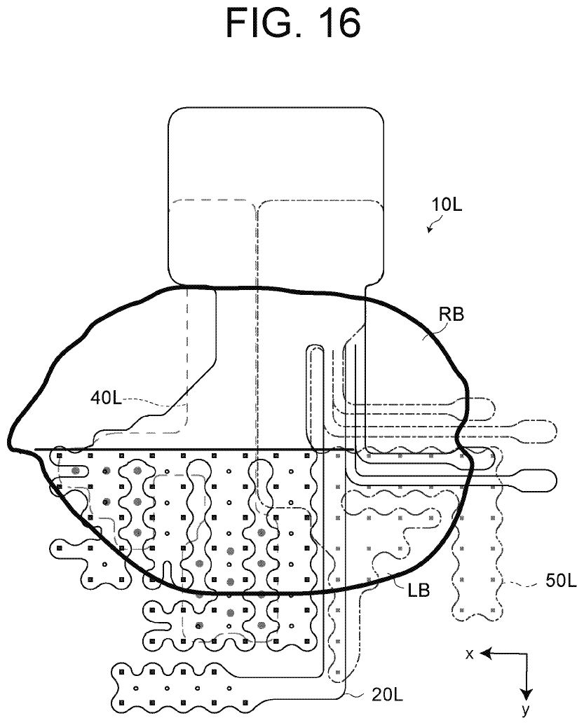

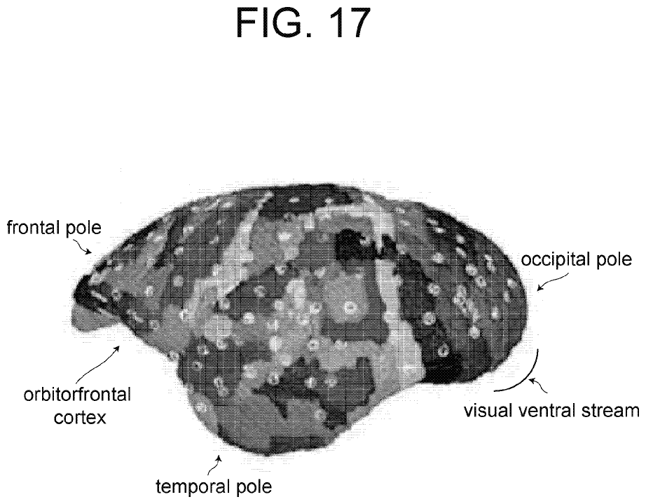

FIG. 16 and FIG. 17 schematically show explanatory diagrams of a state in which the ECoG electrode 10L for left hemisphere according to the embodiment is placed on the living body. FIG. 16 schematically illustrates the ECoG electrode 10L placed on the left hemisphere of the cerebrum as viewed from above. FIG. 17 schematically illustrates an example of the arrangement of the electrodes of the ECoG electrode 10L in the left hemisphere of the cerebrum as viewed from the side.

As shown in FIG. 16 and FIG. 17, the ECoG electrode 10L for left hemisphere is installed so as to fit into the shape of the left hemisphere LB of the cerebrum. Thereby, the entire cerebral cortex of the left hemisphere LB can be covered with high density.

In the same way, the ECoG electrode 10R for right hemisphere is installed so as to fit into the shape of the right hemisphere RB of the cerebrum. Thereby, the entire cerebral cortex of the right hemisphere RB can be covered with high density.

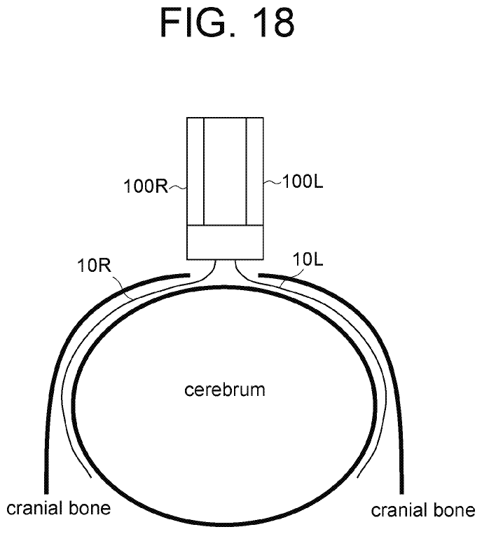

FIG. 18 schematically shows a cross-sectional view of the coronal plane of the cerebrum on which the ECoG electrode 1 according to the embodiments is placed. In FIG. 18, the left side of the brain and the right side of the brain are aligned in the horizontal direction, and the top of the head is in the upper direction.

The case member 100L and the case member 100R are held by sticking back to back using a predetermined holding member, and are arranged vertically near the top of the head of the living body. Here, the case member 100L is configured to accommodate the connector unit of the ECoG electrode 10L for left hemisphere and the case member 100R is configured to accommodate the connector unit of the ECoG electrode 10R for right hemisphere. Each ECoG electrode can cover the entire cerebral cortex of the hemisphere of the cerebrum. Thereby, the electrodes can be placed over the entire cerebral cortex of the left and right hemispheres by sticking the ECoG electrode for left hemisphere and the ECoG electrode for right hemisphere back to back.

As described above, the electrodes can be placed with high density to simultaneously measure the ECoG signals using the ECoG electrode formed of a flexible substrate even in case of a small cerebrum such as a marmoset.

Further, the ground electrode unit and the reference electrode unit are provided on each of the ECoG electrode 20L (20R) for anterior part and the ECoG electrode 50L (50R) for posterior part. Thereby, each of the ECoG electrode 20L (20R) for anterior part and the ECoG electrode 50L (50R) for posterior part can be used alone for measurement. Therefore, the ECoG signals of the anterior part of the cerebral cortex alone can be simultaneously measured or the ECoG signals of the posterior part of the cerebral cortex alone can be simultaneously measured, while minimizing the burden on the living body. For example, the ECoG electrode according to the embodiments can be applied not only when measuring the entire cerebral cortex but also when measuring the visual cortex alone.

Further, the photostimulation electrode 40L (40R) and the ECoG electrode 20L (20R) for anterior part can be stacked, or the photostimulation electrode 40L (40R) and the ECoG electrode 50L (50R) for posterior part can be stacked. Thereby, the ECoG signals of the anterior part of the cerebral cortex alone can be simultaneously measured while applying photostimulation, or the ECoG signals of the posterior part of the cerebral cortex alone can be simultaneously measured while applying photostimulation.

Further, the photostimulation electrode 40L (40R), the ECoG electrode 20L (20R) for anterior part, and the ECoG electrode 50L (50R) for posterior part can be stacked. Thereby, the ECoG signals in the entire hemisphere of the cerebral cortex can be simultaneously measured while applying photostimulation.

Further, the electrodes can be placed at high densities throughout the entire hemisphere of the cerebral cortex by sticking the ECoG electrode 10L for left hemisphere and the ECoG electrode 10R for right hemisphere and arranging the ECoG electrodes on the skull of the living body. Thereby, for example, even in case of a small primate cerebral cortex, the information on brain activity can be simultaneously measured with high accuracy on a large scale.

Further, the photostimulation electrode 40L (40R) is provided separately from the ECoG electrode 20L (20R) for anterior part and the ECoG electrode SOL (50R) for posterior part. Thereby, the noise of the ECoG signals detected in the ECoG electrode 20L (20R) for anterior part and the ECoG electrode SOL (50R) for posterior part can be greatly reduced due to the light emission control in the photostimulation electrode 40L (40R).

Further, the case member 100L (100R) accommodates the ECoG electrode 20L (20R) for anterior part, the ECoG electrode 50L (50R) for posterior part, and the photostimulation electrode 40L (40R) and is disposed on the skull. Thereby, space saving of the connector unit and improvement of noise resistance can be realized, and the noise of the ECoG signals due to the movement of the living body can be significantly reduced. Further, the breakage of the cable, which connects the connector and the external device, due to the movement of the living body or the like can be prevented. And the information on brain activity can be measured for a long time.

### <Brain Activity Acquisition and Control System>

The ECoG electrode 1 according to the embodiments can be applied to a brain activity acquisition and control system.

FIG. 19 shows a block diagram of an example of the configuration of the brain activity acquisition and control system according to the embodiments. In FIG. 19, parts similar to those in FIGS. 1 to 14 are denoted by the same reference symbols, and description thereof is omitted as appropriate.

The brain activity acquisition and control system 200 according to the embodiments can function as a brain activity recording system or a brain activity controlling system. Here, the brain activity recording system is configured to record ECoG signals simultaneously measured at a plurality of positions in the whole or part of the cerebral cortex. The brain activity controlling system is configured to control the brain activity by applying photostimulation to the cerebral cortex.

The brain activity acquisition and control system 200 includes the ECoG electrode 1 and a processor 250.

### (ECoG Electrode 1)

The ECoG electrode 1 includes the ECoG electrode 10L for left hemisphere and the ECoG electrode 10R for right hemisphere. The ECoG electrode 10L for left hemisphere includes the ECoG electrode 20L for anterior part, the ECoG electrode 50L for posterior part, and the photostimulation electrode 40L, and each connector unit is accommodated in the case member 100L fixed on the skull of the living body. The ECoG electrode 10R for right hemisphere includes the ECoG electrode 20R for anterior part, the ECoG electrode 50R for posterior part, and the photostimulation electrode 40R, and each connector unit is accommodated in the case member 100R fixed on the skull of the living body.

In some embodiments, the ECoG electrode 1 includes one of the ECoG electrode 10L for left hemisphere and the ECoG electrode 10R for right hemisphere. In some embodiments, the ECoG electrode for hemisphere included in the ECoG electrode 1 includes one or two of the ECoG electrode for anterior part, the ECoG electrode for posterior part, and the photostimulation electrode.

### (Processor 250)

The processor 250 includes a recording controller 251, a storage 252, an analyzer 253, and a light emission controller 254. Further, the processor 250 includes a connector (not shown), and is electrically connected to each connection terminal of the connector of the ECoG electrode 1 via a cable (not shown).

### (Recording Controller 251)

The recording controller 251 performs control of recording the ECoG signals in the storage 252. The ECoG signals are detected by the plurality of electrodes of the ECoG electrode 1 placed on the surface of the cerebral cortex of the living body, and are received via the cable (not shown). For example, the recording controller 251 converts the received ECoG signal into a first voltage with reference to the ground potential detected by the corresponding ECoG electrode, converts the reference potential into a second voltage with reference to the ground potential detected by the corresponding ECoG electrode, and records the amplitude value of the voltage obtained by subtracting the second voltage from the first voltage in the storage 252 as 16-bit information on brain activity. In some embodiments, conversions into the first voltage and the second voltage are done in the connector provided in the processor 250.

The recording controller 251 records the information on brain activity detected via the corresponding electrode for each detection channel (that is, for each electrode of the ECoG electrode) in the storage 252 in time series. In some embodiments, the information on brain activity is recorded in the storage 252 in association with the light emission control timing of the LED light source by the light emission controller 254 described later.

In some embodiments, the recording controller 251 records the information on brain activity, which is detected through a designated electrode, in the storage 252 in time series. In some embodiments, the recording controller 251 records the information on brain activity, which is detected within a designated period, in the storage 252 in time series. In some embodiments, the recording controller 251 records the information on brain activity, which represents a designated change, in the storage 252 in time series.

### (Analyzer 253)

The analyzer 253 performs predetermined analysis processing based on the information on brain activity recorded in the storage 252. Examples of the analysis processing include processing for estimating an intention of the living body based on the information on brain activity, processing for specifying a state of the brain activity, and the like.

For example, the analyzer 253 performs analysis processing for estimating the intention of the living body based on the information on brain activity recorded in the storage 252. For example, the storage 252 stores in advance an estimation model associated with an electrical characteristic model of information on brain activity for each intention of the living body as a calculation model (decoder) for estimating the intention of the living body. The analyzer 253 selects the above predetermined estimation model that approximates the electrical characteristics of the information on brain activity in the storage 252. And the analyzer 253 estimates that the intention of the living body associated with the selected estimation model corresponds to the intention of the living body represented by the recorded information on brain activity. As the method of estimating based on such multi-measured information on brain activity, for example, the methods disclosed in Japanese Unexamined Patent Application Publication No. 2010-257343 and Japanese Unexamined Patent Application Publication No. 2011-30678 may be employed.

For example, the analyzer 253 performs processing for specifying a state of the brain activity of the living body based on the information on brain activity recorded in the storage 252. For example, the analyzer 253 specifies a state of a predetermined site of the cerebral cortex based on the information on brain activity recorded in the storage 252. In some embodiments, the analyzer 253 specifies the state of the brain activity by searching for a predetermined ECoG signal at a predetermined timing or a time-series pattern of the predetermined ECoG signal. In some embodiments, the analyzer 253 specifies the state of the brain activity by searching for a combination pattern of two or more ECoG signals at a predetermined timing or a time-series pattern of a combination of two or more ECoG signals.

The processor 250 can record the estimation result or the specification result obtained by performing analysis processing by the analyzer 253 in a storage device such as the storage 252 or can output it by an output device (for example, a display, a printer, a speaker, etc.) not shown.

### (Light Emission Controller 254)

The light emission controller 254 controls the light emission timing (light emission start timing, light emission end timing, pulse period, etc.) of the corresponding LED light source for each light emission channel (that is, for each LED light source). In some embodiments, the light emission controller 254 controls the amount of light of the emitted light from the LED light source.

In the case that the LED light source can change the wavelength of the emitted light, the light emission controller 254 can control the wavelength (center wavelength or wavelength range) of the emitted light of the LED light source. For example, the light emission controller 254 controls the LED light source so as to emit light having a wavelength component that activates nerve cells (for example, blue light), or controls the LED light source so as to emit light having a wavelength component that suppresses the activation of nerve cells (for example, orange light).

In some embodiments, the light emission controller 254 controls the LED light source based on the information on brain activity stored in the storage 252.

In some embodiments, the storage 252 stores light emission control information in advance. In the light emission control information, a light emission pattern for each light emission channel is determined. The light emission controller 254 controls each of the plurality of LED light sources in accordance with the light emission pattern determined by the light emission control information stored in the storage 252. The LED light source of the target for light emission emits light when a driving voltage is applied from the processor 250.

The functions of the processor 250 are realized by a processor. The processor includes, for example, a circuit(s) such as, for example, a CPU (central processing unit), a GPU (graphics processing unit), an ASIC (application specific integrated circuit), and a PLD (programmable logic device). Examples of PLD include a simple programmable logic device (SPLD), a complex programmable logic device (CPLD), and a field programmable gate array (FPGA). The processor realizes, for example, the function according to the embodiments by reading out a computer program stored in a storage circuit or a storage apparatus and executing the computer program.

The functions of the processor 250, the recording controller 251, the analyzer 253, and the light emission controller 254, which are described above, can be realized by the processor executing a program stored in the storage 252 or the storage apparatus (not shown), for example. Further, the function of the storage 252 can be realized by a storage apparatus such as a memory or a hard disk.

### First Operation Example

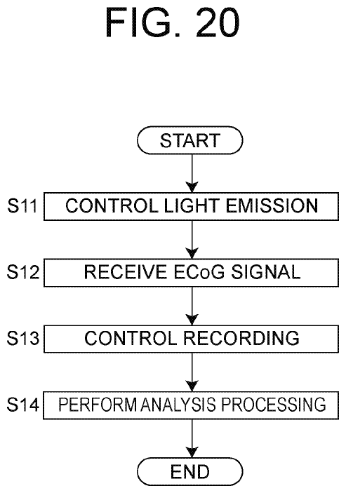

FIG. 20 shows a flow chart of a first operation example of the brain activity acquisition and control system 200 according to the embodiments. For example, the storage 252 stores computer programs for realizing the processing shown in FIG. 20. The processor 250 can execute the processing shown in FIG. 20 by operating in accordance with the computer programs.

### (S11: Control Light Emission)

First, the processor 250 starts controlling light emission of the LED light sources in accordance with the light emission pattern determined by the light emission control information stored in the storage 252.

### (S12: Receive ECoG Signal)

Brain activity is activated by irradiating a desired position of the cerebral cortex of the living body. The ECoG electrode 1 detects the ECoG signals through the electrodes placed at the surface of the cerebral cortex. The processor 250 receives the ECoG signals detected by the ECoG electrode 1 through the cable as described above, in response to irradiation by the LED light sources in step S11.

### (S13: Control Recording)

Next, in the recording controller 251, the processor 250 sequentially generates the information on brain activity based on the ECoG signals received in step S12 and the ground potential and the reference potential detected by the corresponding to the ECoG electrodes, as described above. And the processor 250 (recording controller 251) sequentially records the generated information on brain activity in the storage 252.

The recording controller 251 can sequentially record the information on brain activity in the storage 252 in association with the light emission control timing in step S11.

### (S14: Perform Analysis Processing)

Subsequently, in the analyzer 253, the processor 250 selects the estimation model that approximates the electrical characteristics of the information on brain activity recorded in step S13 from a plurality of estimation models stored in the storage 252. And the processor 250 (analyzer 253) estimates that the intention of the living body corresponding to the selected estimation model is the intention of the living body represented by the recorded information on brain activity described above.

This terminates the operation of the brain activity acquisition and control system 200 (END).

### Second Operation Example

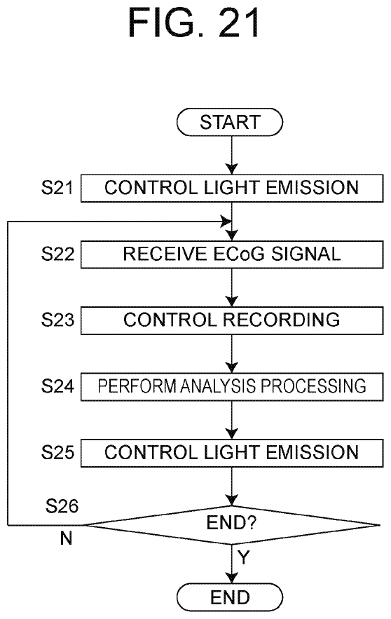

FIG. 21 shows a flow chart of a second operation example of the brain activity acquisition and control system 200 according to the embodiments. For example, the storage 252 stores computer programs for realizing the processing shown in FIG. 21. The processor 250 can execute the processing shown in FIG. 21 by operating in accordance with the computer programs.

### (S21: Control Light Emission)

First, the processor 250 starts controlling light emission of the LED light sources in accordance with the light emission pattern determined by the light emission control information stored in the storage 252.

### (S22: Receive ECoG Signal)

The processor 250 receives the ECoG signals detected by the ECoG electrode 1 through the cable as described above, in response to irradiation by the LED light sources in step S21.

### (S23: Control Recording)

Next, in the recording controller 251, the processor 250 sequentially generates the information on brain activity based on the ECoG signals received in step S22 and the ground potential and the reference potential detected by the corresponding to the ECoG electrodes, as described above. And the processor 250 (recording controller 251) sequentially records the generated information on brain activity in the storage 252.

The recording controller 251 can sequentially record the information on brain activity in the storage 252 in association with the light emission control timing in step S21.

### (S24: Perform Analysis Processing)

Subsequently, in the analyzer 253, the processor 250 specifies the state of the brain activity from the information on brain activity recorded in the storage 252, as described above.

### (S25: Control Light Emission)

Again, the processor 250 performs light emission control on a predetermined LED light source based on the state of the brain activity specified in step S24 by the light emission controller 254.

In some embodiments, the light emission controller 254 performs turn-off control on a desired LED light source based on the state of the brain activity state specified in step S24. In some embodiments, the light emission controller 254 controls the light emission timing (light emission time, turn-off time, pulse width, etc.) of the desired LED light source based on the state of the brain activity specified in step S24. In some embodiments, the light emission controller 254 performs control to change the center wavelength of the emitted light of the desired LED light source based on the state of the brain activity specified in step S24. In some embodiments, the light emission controller 254 starts light emission control for an LED light source different from the LED light source whose light emission is controlled in step S21 based on the state of the brain activity specified in step S24.

### (S26: END?)

Next, the processor 250 determines whether or not to terminates the processing. In some embodiments, the processor 250 determines to terminate the processing when a predetermined instruction is received from the user using an operation unit (not shown). In some embodiments, the processor 250 determines to terminate the processing when a predetermined time has elapsed after the start of the processing. In some embodiments, the processor 250 determines to terminate the processing based on control information stored in the storage 252 in advance.

When it is determined that the processing is to be terminated (S26: Y), the operation of the brain activity acquisition and control system 200 is terminated (END). When it is determined that the processing is not to be terminated (S26: N), the operation of the brain activity acquisition and control system 200 proceeds to step S22.

### [Effects]

The ECoG electrode, the brain activity acquisition and control system, and the brain activity acquisition and control method according to the embodiments will be described.

In the ECoG electrode (1, 10L, 10R, 20L, 20R) according to some embodiments, a plurality of electrodes (Er) and a plurality of wirings (HL) are arranged on a deformable substrate. The plurality of electrodes can be placed at a plurality of positions on a cerebral cortex. The plurality of wirings is electrically connected to each of the plurality of electrodes. The ECoG electrode includes a first connector unit (21L, 21R), one or more electrode units, an electrode unit (29L, 29R) for inferior division of temporal lobe, a ground electrode unit (31L, 31R), and a reference electrode unit (32L, 32R). The first connector unit includes a connector (35L, 35R). The connector has a plurality of connection terminals electrically connectable to the plurality of wirings. The one or more electrode units extends in a first direction (y direction) and has a base connected to the first connector unit. The electrode unit for inferior division of temporal lobe extends in a second direction (x direction) intersecting the first direction via a wiring unit (30L, 30R). The wiring unit (30L, 30R) extends in the first direction and has a base connected to the first connector unit. The ground electrode unit has a base connected to the first connector unit and is used for connecting to the ground potential. The reference electrode unit has a base connected to the first connector unit and is used for detecting the reference signal. The electrode unit (26L, 28L, 26R, 28R) for superior division of temporal lobe is provided on at least one end of the one or more electrode units. The electrode unit for superior division of temporal lobe extends in the second direction and includes an electrode that can be placed in proximity to an electrode arranged in the electrode unit for inferior division of temporal lobe.

According to such a configuration, the ECoG electrode, that is capable of simultaneously measuring the ECoG signals (neural signals) at a plurality of positions on the cerebral cortex, can be provided. In particular, the ECoG signal at the anterior part of the cerebral cortex can be measured alone. Further, the electrode unit for inferior division of temporal lobe extending in the second direction through the wiring unit is arranged with respect to the electrode unit for superior division of temporal lobe extending in the first direction. Thereby, the electrodes can be finely adjusted and arranged at a plurality of positions on the temporal lobe, and the ECoG electrode, that can be buried in a living body having a small cerebrum, can be provided.

In the ECoG electrode according to some embodiments, the one or more electrode units include an electrode unit (22L, 22R) for prefrontal cortex, an electrode unit (23L, 23R) for orbitorfrontal cortex, a first electrode unit (24L, 24R) for prefrontal lobe, and a second electrode unit (25L, 25R) for prefrontal lobe. The electrode unit for superior division of temporal lobe includes one or more wirings extending in the second direction and one or more electrodes electrically connected to the one or more wirings. The electrode unit for superior division of temporal lobe has a base connected to an end of the second electrode unit for prefrontal lobe. The electrode unit for prefrontal cortex has a base connected to the first connector unit and includes one or more wirings extending in the first direction and one or more electrodes electrically connected to the one or more wirings. The electrode unit for orbitorfrontal cortex has a base connected to an end of the electrode unit for prefrontal cortex and includes one or more wirings and one or more electrodes electrically connected to the one or more wirings. The first electrode unit for prefrontal lobe has a base connected to the first connector unit and includes one or more wirings extending in the first direction and one or more electrodes electrically connected to the one or more wirings. The second electrode unit for prefrontal lobe has a base connected to the first connector unit and includes one or more wirings extending in the first direction and one or more electrodes electrically connected to the one or more wirings.

According to such a configuration, the ECoG electrode, that is capable of simultaneously measuring the ECoG signals at a plurality of positions on the anterior part of the small cerebral cortex including a prefrontal cortex, an orbitorfrontal cortex, and a prefrontal lobe, can be provided.

In the ECoG electrode according to some embodiments, a cutout portion (constricted portion 36L, 36R) is formed on an outer edge part between the electrode unit for prefrontal cortex and the electrode unit for orbitorfrontal cortex.

According to such a configuration, the electrodes can be placed with high accuracy without causing the substrate to deflect or the like in a portion where the shape changes in a curved shape in the range from the prefrontal cortex to the orbitorfrontal cortex. In particular, the electrodes can be placed at desired positions on the orbitorfrontal cortex by folding the substrate in the orbitorfrontal cortex located in the inferior region of the cerebral cortex.

The ECoG electrode according to some embodiments includes an electrode unit (27L, 27R) for parietal lobe. The electrode unit for parietal lobe includes one or more wirings extending in the first direction and one or more electrodes electrically connected to the one or more wirings. The electrode unit for parietal lobe has one end connected to the first connector unit.

According to such a configuration, the ECoG electrode, that is capable of simultaneously measuring the ECoG signals at a plurality of positions on the anterior part of the small cerebral cortex further including a part of a parietal lobe, can be provided.

In the ECoG electrodes (1, 10L, 10R, SOL, 50R) according to some embodiments, a plurality of electrodes (Er) and a plurality of wirings (HL) are arranged on a deformable substrate. The plurality of electrodes can be placed at a plurality of positions on a cerebral cortex. The plurality of wirings is electrically connected to each of the plurality of electrodes. The ECoG electrode includes a second connector unit (51L, 51R), an electrode unit (52L, 52R) for visual cortex, an electrode unit (53L, 53R) for visual dorsal stream, an electrode unit (54L, 54R) for occipital lobe, an electrode unit (55L, 55R) for visual ventral stream, a ground electrode unit (61L, 61R), and a reference electrode unit (62L, 62R). The second connector unit includes a connector (65L, 65R). The connector has a plurality of connection terminals electrically connectable to the plurality of wirings. The electrode unit for visual cortex has a base connected to the second connector unit. The electrode unit for visual dorsal stream extends in a third direction (x direction) and has a base connected to the second connector unit. The electrode unit for occipital pole extends in the third direction and has a base connected to the second connector unit. The electrode unit for visual ventral stream extends in a fourth direction (y direction) intersecting the third direction. The electrode unit for visual ventral stream has a base connected to an end of the electrode unit for visual dorsal stream. The ground electrode unit has a base connected to the second connector unit and is used for connecting to the ground potential. The reference electrode unit has a base connected to the second connector unit and is used for detecting the reference signal.

According to such a configuration, the ECoG electrode, that is capable of simultaneously measuring the ECoG signals at a plurality of positions on the posterior part of the small cerebral cortex including a visual cortex, a visual dorsal stream, an occipital pole, and a visual ventral stream, can be provided. In particular, the ECoG signal at the posterior part of the cerebral cortex can be measured alone.

The ECoG electrode according to some embodiments includes an electrode unit (56L, 56R) for parietal lobe. The electrode unit for parietal lobe includes one or more wirings extending in the third direction and one or more electrodes electrically connected to the one or more wirings. The electrode unit for parietal lobe has one end connected to the second connector unit.

According to such a configuration, the ECoG electrode, that is capable of simultaneously measuring the ECoG signals at a plurality of positions on the posterior part of the small cerebral cortex further including a part of a parietal lobe, can be provided.

In the ECoG electrode according to some embodiments, the electrode unit for visual cortex includes a first electrode unit (521L, 521R) for visual cortex, a second electrode unit (522L, 522R) for visual cortex, and a third electrode unit (523L, 523R) for visual cortex. The first electrode unit for visual cortex includes one or more wirings extending in the third direction and one or more electrodes electrically connected to the one or more wirings. The second electrode unit for visual cortex includes one or more wirings extending in the fourth direction and one or more electrodes electrically connected to the one or more wirings. The third electrode unit for visual cortex includes one or more electrodes formed in a region (gap portion 66L) partially surrounded by the first electrode unit for visual cortex and the second electrode unit for visual cortex.

According to such a configuration, the electrodes can be placed with high accuracy without causing the substrate to deflect or the like in a portion where the shape changes in a curved shape in the range from the visual cortex to the visual ventral stream.

The ECoG electrode according to some embodiments includes a photostimulation electrode (40L, 40R), a case member (100L, 100R), and a cover member. The photostimulation electrode includes a plurality of light sources (LED light sources) and a connector unit (42L, 42R). The plurality of light sources can irradiate a plurality of positions on at least the anterior part of the cerebral cortex with light. The connector unit includes a connector having a plurality of terminals for controlling the light sources electrically connected to the plurality of light sources. The case member is configured to accommodate the connector units of the first connector unit and the photostimulation electrode. In the case member, an opening is formed so as to expose at least a plurality of connection terminals. The cover member is configured to cover the opening formed in the case member.

According to such a configuration, the ECoG electrode, that is capable of simultaneously measuring the ECoG signals at the anterior part of the hemisphere of the small cerebral cortex while reducing the noise due to the movement of the living body and the light emission control in the photostimulation electrode, can be provided.

The ECoG electrode according to some embodiments includes a photostimulation electrode (40L, 40R), a case member (100L, 100R), and a cover member. The photostimulation electrode includes a plurality of light sources (LED light sources) and a connector unit (42L, 42R). The plurality of light sources can irradiate a plurality of positions on at least the anterior part of the cerebral cortex with light. The connector unit includes a connector having a plurality of terminals for controlling the light sources electrically connected to the plurality of light sources. The case member is configured to accommodate the connector units of the second connector unit and the photostimulation electrode. In the case member, an opening is formed so as to expose at least a plurality of connection terminals. The cover member is configured to cover the opening formed in the case member.

According to such a configuration, the ECoG electrode, that is capable of simultaneously measuring the ECoG signals at the posterior part of the hemisphere of the small cerebral cortex while reducing the noise due to the movement of the living body and the light emission control in the photostimulation electrode, can be provided.

The ECoG electrode according to some embodiments includes a photostimulation electrode (40L, 40R), a case member (100L, 100R), and a cover member. The photostimulation electrode includes a plurality of light sources (LED light sources) and a connector unit (42L, 42R). The plurality of light sources can irradiate a plurality of positions on at least the anterior part of the cerebral cortex with light. The connector unit includes a connector (41L, 41R) having a plurality of terminals for controlling the light sources electrically connected to the plurality of light sources. The case member is configured to accommodate the connector units of the first connector unit, second connector unit and the photostimulation electrode which are stacked so as not to overlap each other. In the case member, an opening is formed so as to expose at least a plurality of connection terminals of the first connector unit, second connector unit and the plurality of terminals for controlling light sources. The cover member is configured to cover the opening formed in the case member.

According to such a configuration, the ECoG electrode, that is capable of simultaneously measuring the ECoG signals at the hemisphere of the small cerebral cortex while reducing the noise due to the movement of the living body and the light emission control in the photostimulation electrode, can be provided.

The ECoG electrode according to some embodiments includes an electrocorticographic electrode for left hemisphere including any one of the electrocorticographic electrode described above and an electrocorticographic electrode for right hemisphere including any one of the electrocorticographic electrode described above. The electrocorticographic electrode for right hemisphere includes a plurality of electrodes and a plurality of wirings, which are mirror-arranged with respect to the plurality of electrodes and the plurality of wires in the electrocorticographic electrode for left hemisphere.

According to such a configuration, the ECoG electrode, that is capable of simultaneously measuring the ECoG signals at the entire hemisphere of the small cerebral cortex while reducing the noise due to the movement of the living body and the light emission control in the photostimulation electrode, can be provided.

A brain activity acquisition and control system (100) according to some embodiments includes any one of the electrocorticographic electrode described above, a light emission controller (254) controlling the plurality of light sources, and a recording controller (251) recording electrocorticographic signals detected through the plurality of electrodes in storage.

According to such a configuration, the ECoG electrode, that is capable of simultaneously measuring the ECoG signals at a plurality of positions on the small cerebral cortex, can be recorded.

A brain activity acquisition and control system (100) according to some embodiments includes any one of the electrocorticographic electrode described above, a light emission controller (254) controlling the plurality of light sources based on the electrocorticographic signals detected through the plurality of electrodes.

According to such a configuration, the photostimulation can be applied to the cerebral cortex based on the ECoG signals simultaneously measured at the plurality of positions on the small cerebral cortex.

A brain activity acquisition and control method includes a light emission control step of controlling the plurality of light sources, and a recoding control step of recording electrocorticographic signals detected through the plurality of electrodes of any one of the electrocorticographic electrode described above in a storage.

According to such a method, the ECoG signals, that are simultaneously measured at a plurality of positions on the small cerebral cortex, can be recorded.

A brain activity acquisition and control method according to some embodiments includes a detection step of detecting electrocorticographic signals through the plurality of electrodes of any one of the electrocorticographic electrode described above, and a light emission control step of controlling the plurality of light sources based on the electrocorticographic signals detected in the detection step.

According to such a method, the photostimulation can be applied to the cerebral cortex based on the ECoG signals simultaneously measured at the plurality of positions on the small cerebral cortex.

### <Others>

The above-described embodiments are merely examples for carrying out the present invention. Those who intend to implement the present invention can apply any modification, omission, addition, or the like within the scope of the gist of the present invention.

The electrode of each electrode unit in the ECoG electrode according to the embodiments may not be arranged at the corresponding position of the brain lobe. For example, an electrode for prefrontal cortex may be arranged at the prefrontal lobe, or an electrode for prefrontal lobe my be arranged at the prefrontal cortex.

The ECoG electrode according to the embodiments is not limited to the measurement of the ECoG signal in the entire hemisphere. It can be applied alone or in combination to the measurement of the ECoG signal in the hemisphere, the measurement of the ECoG signal in the anterior part, the measurement of the ECoG signal in the posterior part, or photostimulation.

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An electrocorticographic electrode in which a plurality of electrodes that are positionable at a plurality of positions of a cerebral cortex and a plurality of wirings electrically connected to each of the plurality of electrodes are arranged on a deformable substrate, the electrocorticographic electrode comprising:
a first connector structure including a connector having a plurality of connection terminals electrically connectable to the plurality of wirings;
one or more electrode structures extending in a first direction and having a base connected to the first connector structure;
an inferior division of temporal lobe electrode extending in a second direction intersecting the first direction via a wiring structure, the wiring structure extending in the first direction and having a base connected to the first connector structure;
a ground electrode connectable to ground potential and having a base connected to the first connector structure; and
a reference electrode configured to detect a reference signal and having a base connected to the first connector structure, wherein
a superior division of temporal lobe electrode is provided on at least one end of the one or more electrode structures, the for superior division of temporal lobe electrode extending in the second direction and including an electrode configured to be be placed in proximity to the for inferior division of temporal lobe electrode.

2-15. (canceled)

16. The electrocorticographic electrode of claim 1, wherein the one or more electrode structures comprise:
a prefrontal cortex electrode including one or more wirings extending in the first direction and one or more electrodes electrically connected to the one or more wirings, the prefrontal cortex electrode having a base connected to the first connector structure;
an orbitorfrontal cortex electrode including one or more wirings and one or more electrodes electrically connected to the one or more wirings, the orbitorfrontal cortex electrode having a base connected to an end of the prefrontal cortex electrode;
a first prefrontal lobe electrode including one or more wirings extending in the first direction and one or more electrodes electrically connected to the one or more wirings, the first prefrontal lobe electrode having a base connected to the first connector structure; and
a second prefrontal lobe electrode including one or more wirings extending in the first direction and one or more electrodes electrically connected to the one or more wirings, the second prefrontal lobe electrode having a base connected to the first connector structure, wherein
the superior division of temporal lobe electrode includes one or more wirings extending in the second direction and one or more electrodes electrically connected to the one or more wirings, the superior division of temporal lobe electrode having a base connected to an end of the second prefrontal lobe electrode.

17. The electrocorticographic electrode of claim 16, wherein
a cutout portion is formed on an outer edge part of the electrocorticographic electrode between the prefrontal cortex electrode and the orbitorfrontal cortex electrode.

18. The electrocorticographic electrode of claim 16, further comprising:
a parietal lobe electrode including one or more wirings extending in the first direction and one or more electrodes electrically connected to the one or more wirings, the parietal lobe electrode having one end connected to the first connector structure.

19. An electrocorticographic electrode having a plurality of electrodes configured to be placed at a plurality of positions about a cerebral cortex and a plurality of wirings electrically connected to each of the plurality of electrodes are arranged on a deformable substrate, the electrocorticographic electrode comprising:
a second connector structure including a connector having a plurality of connection terminals electrically connectable to the plurality of wirings;
a visual cortex electrode having a base connected to the second connector structure;
a visual dorsal stream electrode extending in a third direction and having a base connected to the second connector structure;
an occipital pole electrode extending in the third direction and having a base connected to the second connector structure;
a visual ventral stream electrode extending in a fourth direction intersecting the third direction, the visual ventral stream electrode having a base connected to an end of the visual dorsal stream electrode;
a ground electrode connectable to ground potential and having a base connected to the second connector structure, and
a reference electrode configured to detect a reference signal and having a base connected to the second connector structure.

20. The electrocorticographic electrode of claim 19, further comprising
a parietal lobe electrode including one or more wirings extending in the third direction and one or more electrodes electrically connected to the one or more wirings, the parietal lobe electrode having one end connected to the second connector structure.

21. The electrocorticographic electrode of claim 19, wherein the visual cortex electrode comprises:
a first visual cortex electrode including one or more wirings extending in the third direction and one or more electrodes electrically connected to the one or more wirings;
a second visual cortex electrode including one or more wirings extending in the fourth direction and one or more electrodes electrically connected to the one or more wirings; and
a third visual cortex electrode including one or more electrodes formed in a region partially surrounded by the first visual cortex electrode and the second visual cortex electrode.

22. The electrocorticographic electrode of claim 1, further comprising:
a photostimulation electrode including a plurality of light sources and a connector structure, the plurality of light sources being configured to irradiate a plurality of positions in at least an anterior part of a cerebral cortex with light, the connector structure including a connector having a plurality of terminals that control the plurality of light sources;
a case configured to accommodate the first connector structure and the connector structure of the photostimulation electrode so as to be capable of being held and in which an opening is formed so that at least the plurality of connection terminals is exposed; and
a cover configured to cover the opening formed in the case.

23. The electrocorticographic electrode of claim 19, further comprising:
a photostimulation electrode including a plurality of light sources and a connector structure, the plurality of light sources being configured to irradiate a plurality of positions in at least an anterior part of the cerebral cortex with light, the connector structure including a connector having a plurality of terminals that control the plurality of light sources;
a case configured to accommodate the second connector structure and the connector structure of the photostimulation electrode so as to be capable of being held and in which an opening is formed so that at least the plurality of connection terminals are exposed; and
a cover configured to cover the opening formed in the case.

24. An electrocorticographic electrode comprising:
a photostimulation electrode including a plurality of light sources and a connector unit, the plurality of light sources being capable of irradiating a plurality of positions in an anterior part of a cerebral cortex with light, the connector unit including a connector having a plurality of terminals for controlling the light sources electrically connected to the light sources;
an electrocorticographic electrode for anterior part including the electrocorticographic electrode of claim 1;
an electrocorticographic electrode for posterior part including the electrocorticographic electrode of claim 19;
a case member configured to accommodate the first connector unit, the second connector unit, and the connector unit of the photostimulation electrode so as to be capable of being held, the first connector unit, the second connector unit, and the connector unit of the photostimulation electrode being stacked so as not to overlap each other, and in which an opening is formed so that at least the plurality of connection terminals of the first connector unit, the plurality of connection terminals of the second connector unit, and the plurality of terminals for controlling the light sources are exposed; and
a cover member configured to cover the opening formed in the case member.

25. The electrocorticographic electrode of claim 22 comprising:
a first electrocorticographic electrode configured to be applied to a left hemisphere; and
a second electrocorticographic electrode configured to be applied to a right hemisphere, wherein
the electrocorticographic electrode configured to be applied to the right hemisphere includes a plurality of electrodes and a plurality of wirings, which are mirror-arranged with respect to the plurality of electrodes and the plurality of wires in the electrocorticographic electrode configured to be applied to the left hemisphere.

26. A brain activity acquisition and control system comprising:
the electrocorticographic electrode of claim 22;
a light emission controller configured to control the plurality of light sources; and
a recording controller configured to record electrocorticographic signals detected through the plurality of electrodes in a storage.

27. A brain activity acquisition and control system comprising:
the electrocorticographic electrode of claim 22; and
a light emission controller configured to control the plurality of light sources based on electrocorticographic signals detected through the plurality of electrodes.

28. A brain activity acquisition and control method comprising:
a light emission control step of controlling the plurality of light sources; and
a recoding control step of recording electrocorticographic signals detected through the plurality of electrodes of the electrocorticographic electrode of claim 22 in a storage.

29. A brain activity acquisition and control method comprising:
a detection step of detecting electrocorticographic signals through the plurality of electrodes of the electrocorticographic electrode of claim 22; and
a light emission control step of controlling the plurality of light sources based on the electrocorticographic signals detected in the detection step.
